(19) United States
(12) Patent Application Publication
(10) Pub. No.: US 2009/0177079 A1
(43) Pub. Date: Jul. 9, 2009

(54) MEDICAL IMAGING SYSTEM HAVING AN INTEGRATED INJECTION DEVICE

(76) Inventors: Ralf Jauster, Stolberg (DE); Michael Friebe, Recklinghausen (DE); Paul Will Coenen, Titz-Rödingen (DE)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/278,329

(22) PCT Filed: Feb. 13, 2007
(86) PCT No.: PCT/US07/62062

§ 371 Date: Nov. 18, 2008

(30) Foreign Application Priority Data
Feb. 14, 2006 (DE) 102006006952.8

(51) Int. Cl. A61B 5/055

(52) U.S. Cl. 600/420

(57) ABSTRACT

Various embodiments of the present invention relate to an MRI system (1) having a magnetic resonance imaging scanner (2) and having an injection device (3), which comprises an injector head (4), for injection of at least one liquid during an MRI examination. According to various embodiments of the invention, provision is made for the injection device (3) to be attached, or to be able to be detachably attached, to the magnetic resonance imaging scanner (2).

### FIELD OF THE INVENTION

The invention relates generally to an MRI system having a magnetic resonance imaging scanner and an injection device, which comprises an injector head, for injection of at least one liquid during an MRI examination.

### BACKGROUND OF THE INVENTION

Particularly in the field of magnetic resonance imaging (MRI) scanning, injection devices are used inject contrast agent such that the MRI device is capable of generating scans, which can be used for diagnostic purposes of internal organs and vessels in a patient. Known injection devices are generally arranged at some distance from the magnetic resonance imaging scanner in the MR room. On the one hand, the aim of this is to attempt to ensure that the operation of the injection device does not adversely affect the image quality or scan quality of the MRI system. On the other hand, the injectors are separated from the magnetic resonance imaging scanner for safety reasons, since the injectors normally contain magnetic materials which are used during operation of the magnetic resonance imaging scanner. However, the distance between the injector device and the magnetic resonance imaging scanner at the same time leads to comparatively long liquid lines being required from the injector head to the patient, and this leads to a correspondingly large amount of liquid having to be injected per patient. Furthermore, the comparatively long lines running from the injection device to the magnetic resonance imaging scanner impede the work of the medical personnel in the MR room during an MRI examination.

The object of the present invention is to avoid the disadvantages which occur with systems known in the prior art.

### SUMMARY OF THE INVENTION

According to various embodiments of the present the invention, the above-mentioned object is achieved in the case of a medical imaging system (such as an MRI system, for example) by attaching and/or detachably attaching the injection device to the medical imaging scanner. Various embodiments of the invention, for the first time provide an MRI system in which the injector device is located immediately adjacent to the magnetic resonance imaging scanner, and/or attached thereto. This results in a fixed association between the injection device and the magnetic resonance imaging scanner. This may further result in a number of significant advantages. First, the attachment to and thus the arrangement of the injection device immediately adjacent to the magnetic resonance imaging scanner means that the injection device does not occupy any significant floor space in the MR room, which would restrict the work of the medical personnel therein. Furthermore, the space which is required for the injection device that is attached to the magnetic resonance imaging scanner is in any case only slightly larger than the amount of space required for the magnetic resonance imaging scanner itself. In addition, the physical proximity between the injector device and the magnetic resonance imaging scanner ensures that the length of the supply lines from the injector head to the patient is relatively short, so that the required amount of injection liquid per patient is thereby reduced significantly. Furthermore, it has surprisingly been found that direct attachment of the injection device to the magnetic resonance imaging scanner does not necessarily result in any adverse effect on the image quality or scan quality during injector operation. In addition the attachment of the injection device to the magnetic resonance imaging scanner means that there is reduced risk of parts of the injector affected and/or “pulled” by the magnetic fields generated by the scanner. This is particularly true when either magnetic materials are shielded or no magnetic materials are used in the injector.

In order to keep the supply lines from the injector head to the patient as short as possible, it is particularly advantageous to attach the injection device to or to arrange it on the magnetic resonance imaging scanner such that the injector head is provided at the end area of the magnetic resonance imaging scanner and is aligned or can be aligned in the tunnel area, or in the extension of the tunnel area of the magnetic resonance imaging scanner, immediately adjacent to the scanner.

Since in some cases a patient has to be moved into and out of the MRI tunnel repeatedly during an MRI examination, it is possible to design the injector head such that it can be pivoted, specifically in such a manner that the head can be pivoted relative to the magnetic resonance imaging scanner. This ensures that, when the injector head is required, it can be moved very close to the patient, and whenever there is no need for liquid injection, the injector head can be pivoted out of the immediate tunnel area. In this context, it is particularly advantageous for it to be possible to pivot the injector head about a first pivoting axis, which extends at least essentially horizontally, to be precise preferably in an angle range from 0 to 360 degrees. The capability to pivot about the first pivoting axis, which extends at least essentially parallel to the tunnel longitudinal axis of the magnetic resonance imaging scanner, makes it possible to ensure that the injector head together with the nozzles that are provided there not only towards the patient but also upward, in order to allow any air in the system to be let out.

As an alternative to pivoting about the first pivoting axis, but preferably in conjunction with the capability to pivot about the first pivoting axis, the injector head can pivot about a second pivoting axis, which extends at least essentially vertically, to be precise in particular in an angle range of more than 90 degrees, preferably in an angle range between 0 and 180 degrees. The capability to pivot about the second pivoting axis makes it possible for the injector head to be pivoted towards the patient in one position and away from the patient in the other limit position, and to be pivoted out of the end area of the scanner, so that the injector does not impede the patient getting onto the patient table and getting off it, and the movement into the tunnel.

In detail, the injection device has an injector housing in which various components and assemblies can be accommodated, as will be described in more detail herein. This injector housing has a part which is attached or can be detachably attached to the MRI, and on which the injector head is mounted such that it can pivot. In this case, the detachable attachment of the injector housing offers the advantage that the entire injection device can be detached and removed when necessary, for example for repair or maintenance purposes, without adversely affecting the magnetic resonance imaging scanner. Furthermore, the detachable attachment offers the advantage that an injection device can be retrofitted to an existing magnetic resonance imaging scanner.

In principle, there are various possible ways to connect the injection device to the magnetic resonance imaging scanner. Since, in general, the attachment should not cause any damage to the housing of the magnetic resonance imaging scanner, it may be preferable in some embodiments to adhesively bond the injector housing directly or indirectly to the magnetic resonance imaging scanner. In order to achieve a detachable attachment between the injector housing and the magnetic resonance imaging scanner, one preferred embodiment provides for at least one attachment part to be attached, preferably adhesively bonded, to the magnetic resonance imaging scanner, and, for detachable attachment purposes, to interact with a further attachment part which is provided on the injector housing. In detail, the connection is then in such a form that a plastic block or a block composed of some other non-magnetic material which can be machined is used as the attachment part, one of whose faces is machined such that it can rest flat on the magnetic resonance imaging scanner, and can be adhesively bonded there. The opposite side is then aligned at least essentially vertically. An attachment means with which the further attachment part on the injector housing interacts is located on this outer face of the attachment part. By way of example, this makes it possible to achieve a plug connection or latching connection, which in particular is detachable, between the attachment part and the injector housing. Furthermore, in should be understood that it is possible to match an outside surface of the injector housing to the shape of the magnetic resonance imaging scanner, so that the injector housing may be directly adhesively bonded to the scanner.

In order to ensure that the injector device interferes as little as possible with the movement area of the medical personnel during the MRI examination, some embodiments provide an injector housing comprising a housing section (which extends vertically in a side area of the MRI housing), and a housing arm (which is angled into the end area of the magnetic resonance imaging scanner and has the injector head attached thereto). Arranging the injector housing at the side may result in reduced interference with the end face of the magnetic resonance imaging scanner, where a large amount of work and movement occurs prior to and/or during an MRI examination. Particularly, in some embodiments, only the injector head is located in this area and, however, can preferably be pivoted out of the area, as described further herein. In this context, it may be particularly advantageous to provide a horizontally-extending housing section between the vertically-extending housing section and the housing arm, so that the vertically-extending housing section can be arranged further away from the front end face of the magnetic resonance imaging scanner.

In various embodiments, the length of the vertically-extending housing section may vary. The housing section may extend into the central area of the magnetic resonance imaging scanner, but in principle may also extend into the upper area of the magnetic resonance imaging scanner, so that the horizontally-extending housing section is moved past the front end face of the magnetic resonance imaging scanner either at the side or else from above. In any case, with the configuration of the horizontally-extending housing section and the housing arm, it is possible for a joint (which allows for rotation about the first pivoting axis) to be provided between the injector head and the housing arm, while the joint (which may also allow for rotation about the second pivoting axis) may also be provided between the horizontally-extending housing section and the housing arm.

In some embodiments of the present invention, a drive device for injection is provided in the injector housing. In such embodiments, the portion of the injector housing which contains the drive device may be arranged in a side area of the magnetic resonance imaging scanner in order to minimize and/or reduce influence resulting from the drive device on the image or scan quality and the risk of magnetic attraction during operation of the magnetic resonance imaging scanner. The arrangement of the drive device in this area of the magnetic resonance imaging scanner is therefore advantageous since the magnetic field which is produced by the magnets is at its lowest in this area. This therefore considerably reduces the risk of magnetic attraction, and any field which is produced by the drive device in any case has only a minor effect on the image quality of the magnetic resonance imaging scanner. This is particularly true when the drive device is a hydraulic drive device, in which case the electrical drive part can also be arranged in the side area of the magnetic resonance imaging scanner and the nozzles which are provided in the injector head can be operated by appropriate application of pressure by means of hydraulic fluid, that is to say, in the end, no electrical drive means are provided in the area of the injector head. Apart from this, it is self-evident that a pneumatic drive device can also be provided, instead of a hydraulic drive device.

In order to ensure that the forces and moments caused by the drive device that is provided in the housing have as little effect as possible on the magnetic resonance imaging scanner, provision is on the one hand made for the drive device to be provided in the lower area of the injector housing. On the other hand, it is a good idea for the injector housing to stand on the ground when in the attached state, so that the weight forces from the drive device are introduced directly into the floor.

In order to avoid liquid, data and/or supply lines hanging around in the area of the injector device, one particular refinement of the present invention provides for the abovementioned lines to be accommodated in the injector housing and preferably also in the housing of the injector head, to be precise in particular in such a manner that they cannot be seen from the outside.

However, in the case of the present invention, the injector housing not only offers the advantage that the drive apparatus can be accommodated in it, but at least one accommodation area can also be provided for liquid and/or nozzles to be injected. The accommodation area, in which other objects can of course also be positioned, can preferably be closed via a flap or a cover.

Finally, in conjunction with the injector apparatus according to the invention, it is possible for the housing of the injector head and/or of the injector housing to be composed of a non-magnetic material. The entire injector device including all of the components is preferably composed of a non-magnetic material or a material which cannot be magnetized, in order to satisfy the safety requirements, to avoid any shielding, and to avoid adversely affecting the image quality.

### BRIEF DESCRIPTION OF THE DRAWINGS

Having thus described various embodiments of the invention in general terms, reference will now be made to the accompanying drawings, which are not necessarily drawn to scale, and wherein:

FIG. 1 shows a perspective front view of an MRI system with a magnetic resonance imaging scanner and an injection device attached thereto, according to one embodiment of the present invention;

FIG. 2 shows a view, corresponding to FIG. 1, of an alternate embodiment of an MRI system, according to one embodiment of the present invention;

FIG. 3 shows a view, corresponding to FIG. 2, of a further alternate embodiment of an MRI system, according to one embodiment of the present invention;

FIG. 4 shows a view of an injection device according to one embodiment of the present invention;

FIG. 5 shows a view of the injection device from FIG. 4, looking in the direction of the arrow V from FIG. 4, according to one embodiment of the present invention;

FIG. 6 shows a perspective view of a different embodiment of an injection device, according to one embodiment of the present invention;

FIG. 7 shows a view of an alternate embodiment of an injection device according to one embodiment of the present invention;

FIG. 8 shows a perspective view of an attachment part according to one embodiment of the present invention;

FIG. 9 shows a schematic view of the attachment part attached to a magnetic resonance imaging scanner, according to one embodiment of the present invention;

FIG. 10 shows a schematic view of an MRI system, according to one embodiment of the present invention, with an MR room and a control room; and

FIG. 11 shows a view, corresponding to FIG. 10, of an alternate embodiment of an MRI system, according to one embodiment of the present invention.

### DETAILED DESCRIPTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

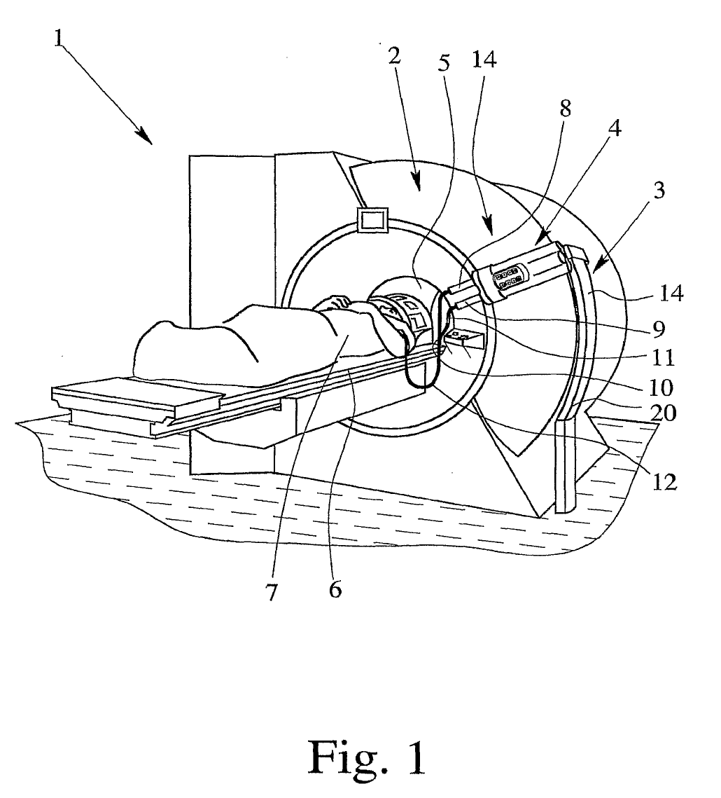

FIGS. 1-3 each show MRI systems 1 which have a magnetic resonance imaging scanner 2 and an injection device 3 for injection of at least one liquid during an MRI examination or other medical procedure. The injection device 1 may comprise an injector head 4 for this purpose. In the illustrated embodiments, the magnetic resonance imaging scanners 2 each have a tunnel 5 with a patient table 6. In the case of the embodiment illustrated in FIG. 1, a patient 7 is located on the patient table 6. The patient table 6 can, in some embodiments, be moved into and out of the tunnel 5.

It should be understood that various embodiments of the present invention are not restricted to the illustrated “closed” MRI appliances. MRI appliances of an open form (i.e. “open MRI's,” for example) are also covered by various embodiments of the present invention. In general, the present invention applies to all MRI magnet systems, superconducting, permanent or resistive, in a closed or open form, with a vertical or horizontal magnetic-field alignment as well as with an alignment between the horizontal and vertical. Apart from this, it is self-evident that the magnetic resonance imaging scanner 2 has, in a manner known per se, magnets and coils as well as other devices which are required for MRI operation.

It should be further understood that the various embodiments of the present invention may be used not only for an MRI system but in a similar fashion for a CT system. In such embodiments, all of the features mentioned herein should be understood to relate to a computed-tomography scanner rather than a magnetic resonance imaging scanner.

As can be seen from the various individual figures shown herein, the injector head 4 comprises two nozzles 8, 9 on the outlet side, via which at least one of a contrast agent on the one hand and a saline solution on the other hand can be supplied to the patient. For this purpose, the nozzles 8, 9 can be connected via a Y-adapter 10 and/or various intermediate flexible tubes 11 to a catheter 12, through which the contrast agent and/or the saline solution can be injected during the MRI examination.

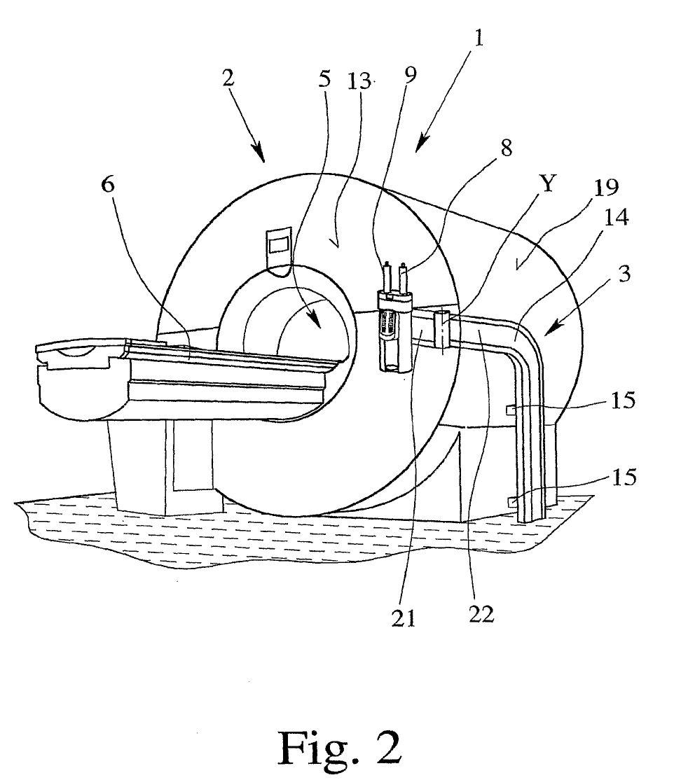

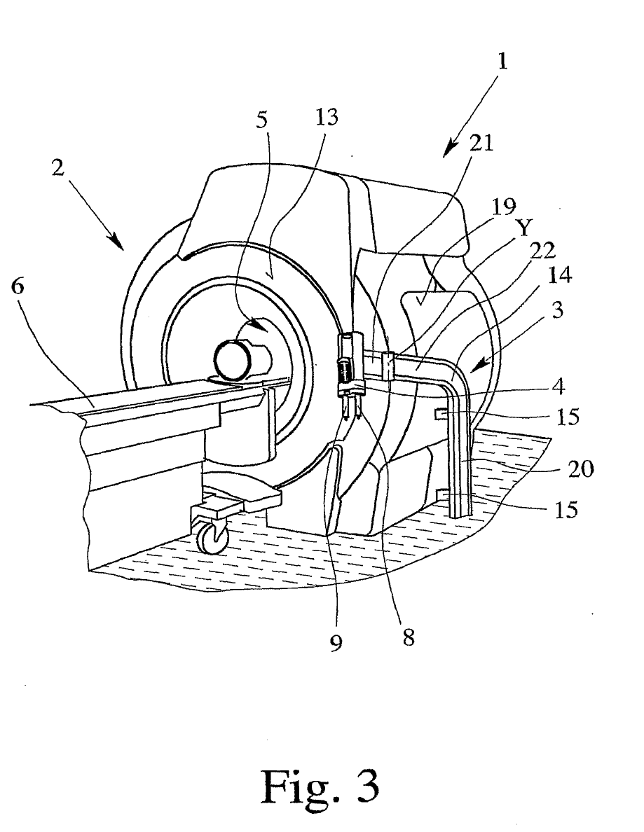

As shown herein, the injection device 3 is operably engaged with the magnetic resonance imaging scanner 2 either non-detachably or else detachably. The various embodiments described herein are designed such that the attachment and arrangement are provided in such a manner that the injector head 4 is provided adjacent to the front end face 13 of the magnetic resonance imaging scanner 2 and is aligned or can be aligned from the side in the direction of the tunnel opening, in the radial direction with respect to the tunnel opening of the magnetic resonance imaging scanner. While FIG. 1 shows the injection position, FIGS. 2 and 3 each show a position in which the injector head 4 is not in use. In FIG. 2, the injector head 4 is pivoted upwards for venting, while it has been pivoted downwards in FIG. 3.

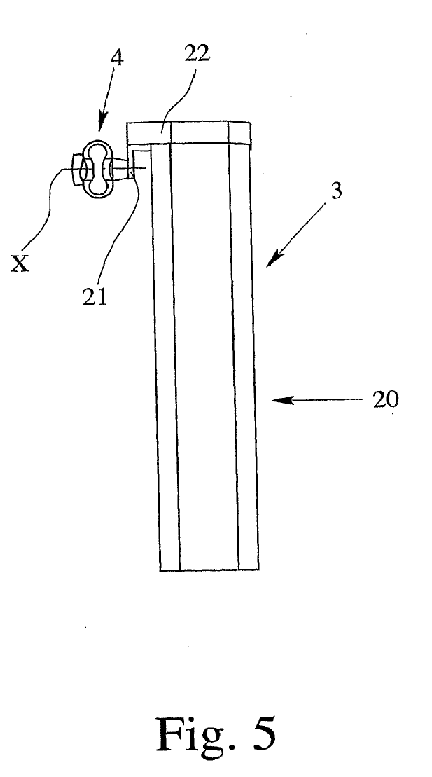

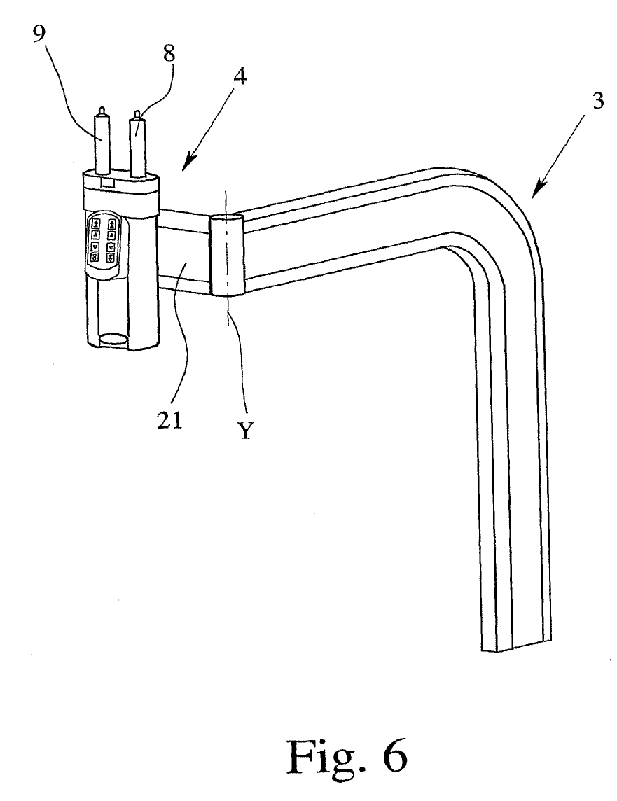

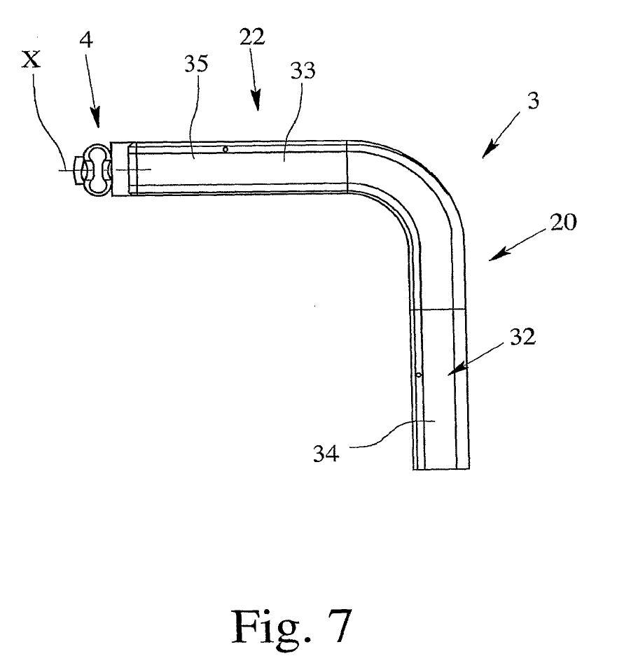

In order to allow the injector head 4 to pivot (as can be seen from a comparison of FIGS. 1 to 3), a substantially horizontal first pivoting axis X is provided in a joint, which is not illustrated in detail. The pivoting axis X can be seen in particular in FIG. 5. The joint for the first pivoting axis X is in this case designed in such a manner that the injector head 4 can be pivoted continuously variably in an angle range from 0 to 360 degrees. Smaller angle ranges are also in principle possible. Furthermore, the injection devices 3 which are illustrated in FIGS. 2 and 3 (as well as in FIGS. 6 and 7) have a second pivoting axis Y, which extends at a substantially right angle to the first pivoting axis X. In the various illustrated embodiments, the second pivoting axis Y extends substantially vertically relative to a floor of the MR room. Although this is not illustrated in detail, the injector head 4 can pivot continuously variably about the second pivoting axis Y in an angle range from 0 to 180 degrees. In principle, it is even possible for the injector head 4 to pivot continuously variably about the second pivoting axis Y up to 270 degrees, in order to pivot the injector head 4 into a side area of the magnetic resonance imaging scanner 2.

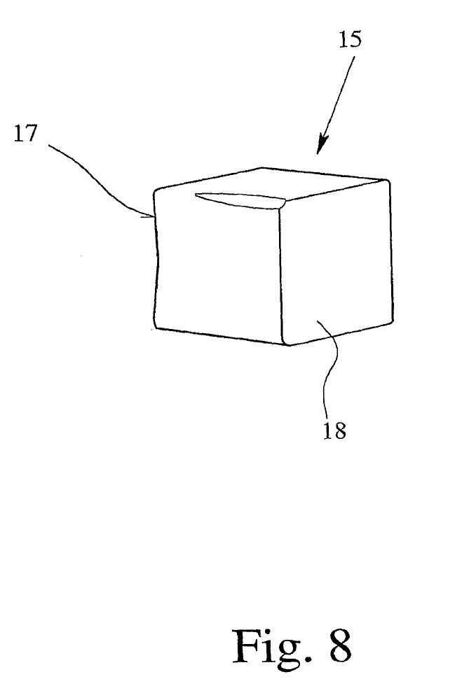

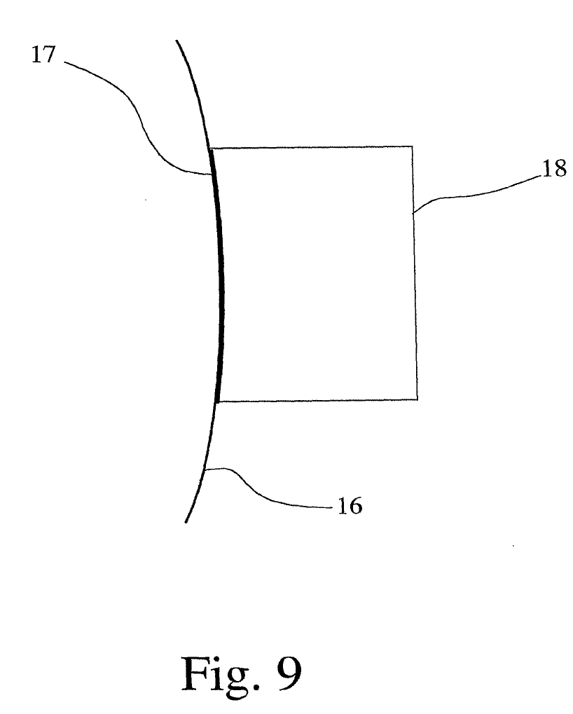

In addition to the injector head 4 which can pivot, the injection device 3 has an injector housing 14 which is attached or else can be detachably attached to the magnetic resonance imaging scanner 2, on which the injector head 4 is mounted, such that it can pivot. In order to avoid damage to the outer surface or the housing of the magnetic resonance imaging scanner 2, the injector housing 14 may be adhesively bonded to the magnetic resonance imaging scanner 2. In such embodiments, illustrated generally in FIG. 1, the injector housing 14 is directly adhesively bonded to the magnetic resonance imaging scanner 2. In such embodiments, the injector housing 14 may be provided with a shape that is substantially matched to that of the scanner 2 on the side facing the magnetic resonance imaging scanner 2. In contrast, in the embodiments illustrated generally in FIGS. 2 and 3, at least one attachment part 15, illustrated more particularly in FIGS. 8 and 9, may be provided on the magnetic resonance imaging scanner 2. More particularly, in some embodiments, the attachment part 15 may be adhesively bonded to the housing 16 of the magnetic resonance imaging scanner 2. In some embodiments, the attachment part 15 may include, but is not limited to: a plastic block, whose rear face 17 may be machined (if necessary) such that the rear face 17 rests flat on the housing 16 of the magnetic resonance imaging scanner 2, and therefore may be adhesively bonded thereto. The rear face 17 may be configured to substantially match the housing 16 of the magnetic resonance imaging scanner 2 in such a manner that the front face 18 is aligned essentially vertically. This defined arrangement and alignment make it possible to provide a detachable latching connection or plug connection between the attachment part 15 and the injector housing 14, in which case corresponding latching mechanisms or plug mechanisms, which are not illustrated in detail, may then be provided on the attachment part 15 and on corresponding portions of the injector housing 14. Furthermore, it should be understood that the number and arrangement of the attachment parts 15 depend on the respective relationships between the magnetic resonance imaging scanner 2 as well as the larger embodiment and weight of the injection device 3.

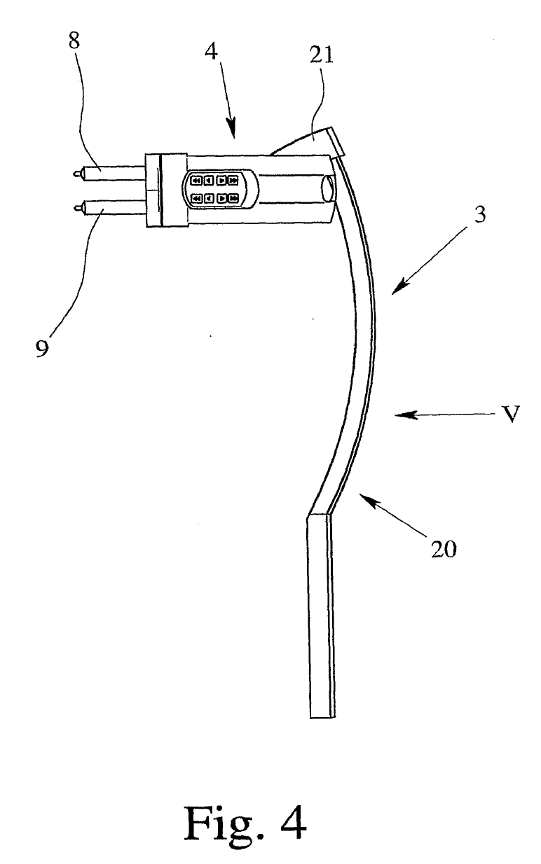

As shown in the various figures presented herein, the injector housing 14 may comprise a housing section 20 extending substantially vertically in the side area 19 of the magnetic resonance imaging scanner 2. Furthermore, the injector housing 14 may further comprise a housing arm 21 which is angled into the front end area 13 of the magnetic resonance imaging scanner 2 and has the injector head 4 operably engaged therewith. Although, in principle, it is possible for the housing arm 21 (which may be provided at the end of the housing arm 21) to project directly from the vertical housing section 20, in some embodiments a horizontally-extending housing section 22 is adjacent to the vertically-extending housing section 20. Thus, as shown in the figures presented herein, the housing arm 21 may be operably engaged with the horizontally-extending housing section 22. While the horizontally-extending housing section 22 is shown in a relatively short configuration in the embodiments of the injector device 3 illustrated in FIGS. 1, 4 and 5, the horizontal housing section 22 may also be provided in a relatively long configuration as shown generally in FIGS. 2, 3, 6 and 7. In various embodiments, the horizontal housing section 22 may be provided in a length that corresponds substantially to that of the vertically extending housing section 20. In such embodiments, the first pivoting axis X is provided between the injector head 4 and the housing arm 21, while the second pivoting axis Y and the joint which allows for rotation about the second pivoting axis Y are provided between the horizontally-extending housing section 22 and the housing arm 21.

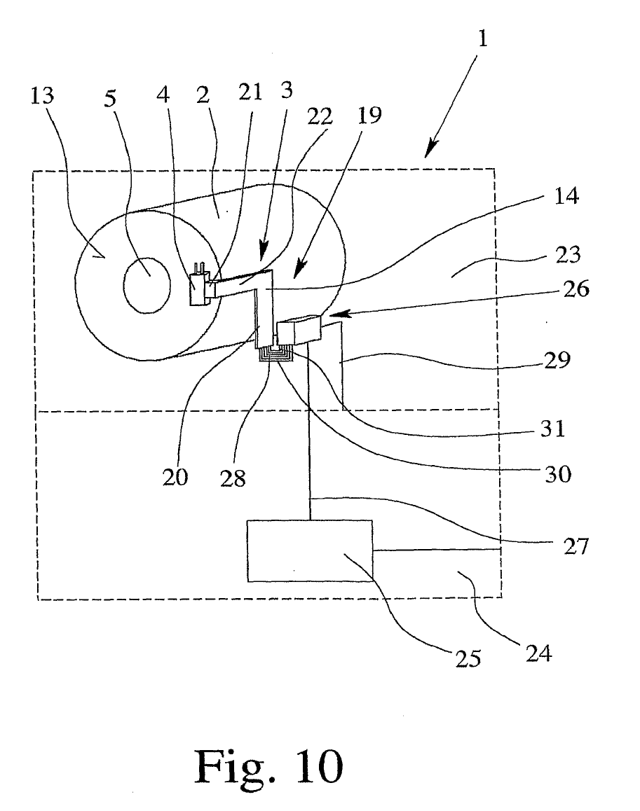

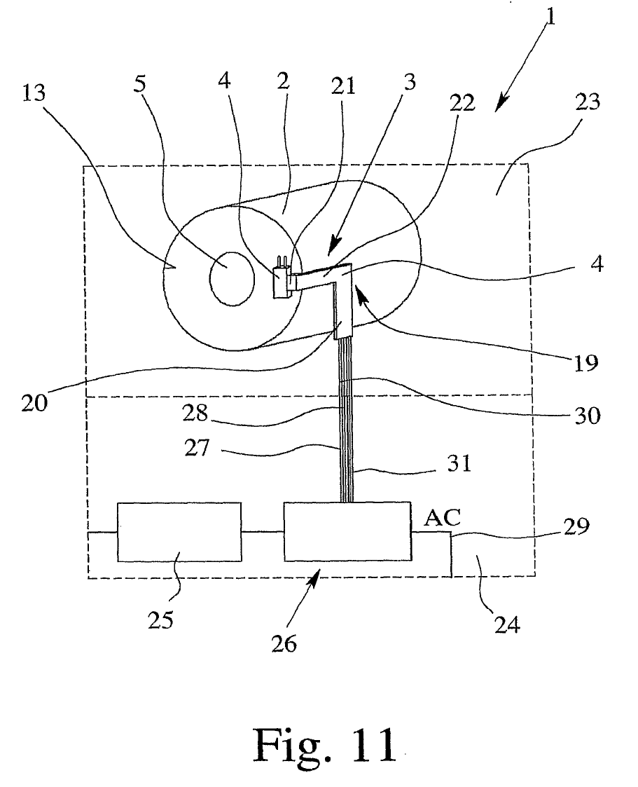

FIGS. 10 and 11 each show an MRI system 1 in which the magnetic resonance imaging scanner 2 and the injection device 3 are each arranged in an MR room 23. A system controller 25 is located in a control room 24. In both cases, the injection device 3 has an associated drive device 26 for injection. The difference between the embodiments shown in FIG. 10 and FIG. 11 is that the drive device 26, which is driven by the system control via an optical waveguide 27, is located in the MR room 23 in the embodiment shown in FIG. 10, while it is located in the control room 24 in the embodiment shown in FIG. 11. However, it should be understood that the system controller 25 and the drive device 26 need not necessarily be connected via an optical waveguide 27. In principle, any type of conductor can be provided for establishing communication between the system controller 25 and the drive device 26.

In both embodiments (i.e. those shown in FIGS. 10 and 11), the drive device 26 may comprise an hydraulic drive device 26, in which the injector head and the mechanism provided therein are driven hydraulically via one or more hydraulic lines 28. As illustrated in FIG. 10, the drive device 26 may be arranged in the side area 19 of the magnetic resonance imaging scanner 2, and alongside the vertically-extending housing section 20. In some alternative embodiments, however, the drive device 26 may also be arranged, in the vertically-extending housing section 20, and in particular in a lower portion thereof. The particular technical advantage of the arrangement shown generally in FIG. 10 is that the length of the hydraulic lines 28 may be reduced considerably in comparison to the embodiment shown in FIG. 11.

In other embodiments, an alternating-current (AC) supply line 29 may be connected to the input side of the drive device 26 for an electrical drive, and the system controller 25 may be connected to its input side by the optical waveguide 27 that has been mentioned herein. An optical waveguide 30 for signal transmission, the hydraulic lines 28 and a power supply line 31 (direct current (DC), for example)) may be also provided on the output side.

Although, as can be seen generally from FIGS. 10 and 11, a plurality of lines are passed through the injector housing 14, these may not be visible from the outside, as is evident in particular from FIGS. 1 to 7, but are accommodated in a concealed form in the injector housing 14. In this case, FIG. 7 indicates that a plurality of accommodation areas 32, 33 are provided in the injector housing 14. Each of the accommodation areas 32, 33 can be opened by means of a flap 34, 35. In this case, in the embodiment shown in FIG. 7, the drive device 26 together with the electrical drive may be located in the accommodation area 32, while the accommodation area 33 may be used to accommodate nozzles, saline solution and/or contrast agents. Furthermore, in some embodiments, the various AC supply lines 29, optical waveguide 30 for signal transmission, hydraulic lines 28 and/or power supply lines 31 that have already been mentioned herein may also be passed through the accommodation area 33.

Furthermore, particularly in the case of embodiments in which the drive device 26 is provided in the injector housing 14, it is important for the injector housing 14 (with its vertically-extending housing section 20) to stand on a floor or other horizontal platform such that the weight forces of the injector housing 14 may be adequately absorbed.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### LIST OF EXEMPLARY REFERENCE SYMBOLS

1 MRI system
2 Magnetic resonance imaging scanner
3 Injection device
4 Injector head
5 Tunnel
6 Patient table
7 Patient
8 Nozzle
9 Nozzle
10 Y adapter
11 Intermediate flexible tube
12 Catheter
13 End face
14 Injector housing
15 Attachment part
16 Housing
17 Rear face
18 Front face
19 Side area
20 Vertical housing section
21 Housing arm
22 Horizontal housing section
23 MR room
24 Control room
25 System controller
26 Drive device
27 Optical waveguide
28 Hydraulic lines
29 Power supply line
30 Optical waveguide
31 Power supply line
32 Accommodation area
33 Accommodation area
34 Flap
35 Flap
X First pivoting axis
Y Second pivoting axis

1. A medical imaging system comprising:
a magnetic resonance imaging scanner; and
an injection device comprising an injector head, for injection of at least one liquid during an MRI examination, wherein
the injection device is attached, or detachably attached, to a side area of the magnetic resonance imaging scanner, and wherein
the injector head is attached to the magnetic resonance imaging scanner such that the injector head is capable of pivoting with respect to the magnetic resonance imaging scanner at least about a first pivoting axis and a second pivoting axis extending substantially perpendicular to the first pivoting axis.

2. The medical imaging system as claimed in claim 1, wherein the injection device is attached to and arranged on the magnetic resonance imaging scanner such that the injector head is provided at an end of the magnetic resonance imaging scanner and is aligned or can be aligned at least essentially in the radial direction with respect to a tunnel opening defined in the magnetic resonance imaging scanner.

3. The medical imaging system as claimed in claim 1, wherein the injector head can be pivoted relative to the magnetic resonance imaging scanner.

4. The medical imaging system as claimed in claim 1, wherein the injector head can be pivoted about the first pivoting axis, which extends at least essentially horizontally, and about the second pivoting axis, which extends at least essentially vertically.

5. The medical imaging system as claimed in claim 1, wherein the injector head can be pivoted through an angle range from 0° to 360° about the first pivoting axis, and in an angle range of more than 90°, in particular from 0 to 180°, about the second pivoting axis.

6. The medical imaging system as claimed in claim 1, wherein the injection device comprises an injector housing attached, or detachably attached, to the magnetic resonance imaging scanner.

7. The medical imaging system as claimed in claim 1, wherein the injection device is adhesively bonded to the magnetic resonance imaging scanner.

8. The medical imaging system as claimed in claim 6, wherein at least one attachment part is attached, preferably adhesively bonded, to the magnetic resonance imaging scanner, and, for detachable attachment purposes, interacts with a further attachment part, which is provided on the injector housing.

9. The medical imaging system as claimed in claim 8, wherein the attachment part and the further attachment part cooperate to form a plug connection and/or a latching connection.

10. The medical imaging system as claimed in claim 8, wherein the attachment part comprises a plastic block, which can be machined at the MRI end, and/or a block composed of a non-magnetic material.

11. The medical imaging system as claimed in claim 6, wherein the injector housing comprises a housing section, which extends vertically in a side area of the magnetic resonance imaging scanner, and a housing arm, which is angled into the area of the end face of the magnetic resonance imaging scanner and wherein the injector head is operably engaged with the housing arm.

12. The medical imaging system as claimed in claim 11, wherein a horizontally extending housing section is provided between the vertically extending housing section and the housing arm.

13. The medical imaging system as claimed in claim 12, further comprising a first joint disposed between the injector head and the housing arm configured for pivoting about the first pivoting axis and/or a second joint disposed between the horizontally extending housing section and the housing arm configured for pivoting about the second pivoting axis.

14. The medical imaging system as claimed in claim 1, wherein a drive device for injection is contained in the injector housing, and wherein a portion of the injector housing which contains the drive device is arranged in a side area of the magnetic resonance imaging scanner.

15. The medical imaging system as claimed in claim 14, wherein the drive device comprises at least one of a hydraulic drive device and a pneumatic drive device.

16. The medical imaging system as claimed in claim 14, wherein the drive device is disposed in a lower area of the injector housing and wherein, preferably, the injector housing stands on the floor when in the attached state.

17. The medical imaging system as claimed in claim 6, wherein a plurality of liquid and/or supply lines are disposed substantially within the injector housing and/or substantially within a housing of the injector head.

18. The medical imaging system as claimed in claim 6, wherein at least one accommodation area for the liquid and/or nozzle to be injected is provided in the injector housing.

19. The medical imaging system as claimed in claim 18, wherein the accommodation area comprises a closeable flap.

20. The medical imaging system as claimed in claim 6, wherein a housing of the injector head and the injector housing are composed of a non-magnetic material.
